(19) United States
(12) Patent Application Publication
(10) Pub. No.: US 2024/0350216 A1
(43) Pub. Date: Oct. 24, 2024

(54) ADJUSTABLE CLAMPING ARRANGEMENTS FOR SURGICAL ARMS

(71) Applicant: MOMENTIS SURGICAL LTD., Or-Yehuda (IL)

(72) Inventor: Eyal MAIMON, Kfar Adumim (IL)

(21) Appl. No.: 18/687,337

(22) PCT Filed: Sep. 1, 2022
(86) PCT No.: PCT/IB2022/058212

§ 371 Date: Feb. 28, 2024

Related U.S. Application Data
(60) Provisional application No. 63/239,424, filed on Sep. 1, 2021.

(51) Int. Cl. A61B 34/37; A61B 17/00; A61B 90/57

(52) U.S. Cl. CPC A61B 34/37; A61B 2017/00725; A61B 2090/571

(57) ABSTRACT

A surgical apparatus comprises a motor-control unit for controlling one or more surgical arms. The motor-control unit comprises an arm-receiving volume for each of the surgical arms, and an array of clamping-hammers aligned with each arm-receiving volume and configured to apply pressure on the surgical arm to secure it. Each clamping-hammer includes a clamping member configured to displace between a distal position spaced from the arm-receiving volume and a proximal position adjacent to the arm-receiving volume, and a pressure member fitted to the clamping member and configured for coming in contact with a contact area of the arm. The pressure member is configured to displace with respect to the clamping member, thereby allowing regulation of the pressure applied to the contact area.

### FIELD OF THE INVENTION

The present invention relates to surgical devices comprising one or more mechanical arms that are articulated and/or flexible, and in particular to adjustable and calibratable clamping arrangements for retaining the arms within the surgical devices, and devices and methods for calibrating the clamping arrangements.

### BACKGROUND

It is well established that there are benefits of minimally invasive surgery. Instruments for such surgery typically have a surgical end effector located at the distal end of an articulated surgical arm (preferably with minimum diameter) that is inserted through a small opening (e.g., body wall incision, natural orifice) to reach a surgical site. In some instances, surgical instruments can be passed through a cannula and an endoscope can be used to provide images of the surgical site.

Surgical instruments have been developed that utilize an end effector (e.g., a surgical tool such as for tissue fusing or cutting, or a measurement tool) for convenience, accuracy, and wellbeing of the subject. In some cases, articulated surgical arms have one or more bending portions which are controlled remotely using various input devices (e.g., hand and foot controls) to ultimately control the location of the end effector and change its orientation with reference to the surgical arm's longitudinal axis.

Motor-control units include gears which mesh with gears of surgical arms for precise control of the arms. The current state of the art is lacking devices and methods for retaining the arms within the motor-control units which offer precise and controllable transmission of forces between the gears. There is therefore a need for adjustable and/or calibratable clamping arrangements for retaining the arms within the motor-control units with precise transmission of forces between the gears while still allowing easy exchange of the surgical arms.

### SUMMARY OF THE INVENTION

According to embodiments disclosed herein, a surgical apparatus comprises a motor-control unit for controlling one or more surgical arms. The motor-control unit comprises, for each one of the one or more surgical arms: (i) an arm-receiving volume shaped to receive a portion of a respective one of the one or more surgical arms, and (ii) an array of clamping-hammers aligned with the arm-receiving volume and configured for applying pressure to said portion of the surgical arm to thereby secure said portion in said volume. Each clamping-hammer comprises: (a) a clamping member configured for displacement with respect to said volume at least between a distal position spaced from said arm-receiving volume and a proximal position adjacent to said arm-receiving volume; and (B) a pressure member fitted to the clamping member and configured for coming in contact with a respective contact area of said portion of the surgical arm, wherein said pressure member is configured to displace with respect to the clamping member, thereby allowing regulation of the pressure applied to said respective contact area.

In some embodiments, the surgical apparatus can additionally comprise a closure flap in communication with one or more clamping members, wherein pivoting the closure flap to a closed position is effective to place the one or more clamping members in respective proximal positions. In some such embodiments, it can be that each clamping-hammer further comprises a regulating member fitted to the clamping member and configured to regulate the displacing of a respective pressure member, and when the one or more clamping members are in respective proximal positions and the closure flap is in the closed position, regulating members respective of the one or more clamping members are exposed.

In some embodiments, the displacing of a pressure member can be regulable by reorientation of the respective regulating member.

In some embodiments, the closure flap can be detachably coupled to each of the one or more clamping members.

In some embodiments, the regulating member can include a threaded element, and rotation of the threaded element is effective to regulate the displacing of a respective pressure member.

In some embodiments, the pressure applied to said portion of the surgical arm can be set by a reorientation of the regulating member performed with an instrumented calibration apparatus seated in the arm-receiving volume.

In some embodiments, the array of clamping-hammers can include at least three clamping-hammers, and the at least three clamping-hammers can be jointly regulable to apply substantially equal pressures to said portion of the surgical arm at respective contact areas.

In some embodiments, the surgical apparatus can further comprise an array of gears arranged in the arm-receiving volume to be in geared contact with corresponding surgical-arm gears of the respective surgical arm, wherein each clamping-hammer of at least a plurality of clamping-hammers is aligned with a corresponding arm-receiving-volume gear.

In some embodiments, the regulation of the pressure applied to a respective contact area can be effective to regulate a force of engagement of a surgical-arm gear of the respective surgical arm with a corresponding motor gear disposed within the arm-receiving volume.

According to embodiments disclosed herein, a motor-control unit for controlling one or more surgical arms comprises, for each one of the one or more surgical arms: (a) an arm-receiving volume shaped to receive a portion of the respective surgical arm, and (b) an array of clamping-hammers aligned along the arm-receiving volume, each clamping-hammer being orientable to a respective closed state and biased, when in said respective closed state, to contact a surgical arm seated in the arm-receiving volume and to apply a pressure thereto, wherein the pressure is regulable by reorienting a pressure-regulating portion of the clamping-hammer.

In some embodiments, each clamping-hammer of the array of clamping-hammers can comprise: (i) a base member anchored to the motor-control unit, (ii) a clamping member pivotably attached to the base member, and (iii) a lever member pivotably attached to each of the base member and the clamping member, and biased, when in a closed state, to transfer a clamping force to the clamping member, the clamping force being effective, when the respective surgical arm is seated in the arm-receiving volume, to cause an arm-contacting portion of the clamping member to contact the surgical arm and to apply a pressure thereto.

In some embodiments, the reorienting of the pressure-regulating portion can be effective to reorient the clamping member. In some embodiments, the reorienting of the pressure-regulating portion can be effective to reorient the arm-contacting portion.

In some embodiments, the motor-control unit can additionally comprise a closure flap in communication with one or more lever members, and pivoting the closure flap to a closed position can be effective to place the one or more lever members in respective closed states.

In some embodiments, it can be that when the one or more lever members are in their respective closed states and the closure flap is in the closed position, the pressure-regulating portions respective of corresponding clamping members are exposed.

In some embodiments, the closure flap can be detachably coupled to each of the one or more lever members.

In some embodiments, the reorientation of the pressure-regulating portion can include rotation of a threaded element.

In some embodiments, the pressure applied to the surgical arm can be set by a reorientation of the pressure-regulating portion performed while an instrumented calibration apparatus is seated in the arm-receiving volume.

In some embodiments, the array of clamping-hammers can include at least three clamping-hammers, and the at least three clamping-hammers can be jointly regulable to apply substantially equal pressures to the surgical arm.

In some embodiments, the motor-control unit can further comprise, for each one of the one or more surgical arms, an array of gears arranged in the arm-receiving volume to be in geared contact with corresponding surgical-arm gears of the surgical arm, and each clamping-hammer of at least a plurality of clamping-hammers can be aligned with a corresponding motor-unit gear in the arm-receiving volume.

In some embodiments, the regulation of the pressure applied to the surgical arm by each clamping-hammer can be effective to regulate a force of engagement of a surgical-arm gear of the surgical arm with a corresponding motor gear disposed within the arm-receiving volume.

According to embodiments disclosed herein, an apparatus for use with a surgical-arm motor-control unit that comprises an array of clamping-hammers aligned along an arm-receiving volume comprises, wherein each clamping-hammer is closable to a respective closed state so as to contact a surgical arm seated in the arm-receiving volume and to apply a pressure thereto, comprises: (a) an elongate element shaped to be seated in the arm-receiving volume; and (b) an array of force-measuring elements, wherein for at least one of the clamping-hammers, when the apparatus is seated in the arm-receiving volume and the clamping-hammer is in its respective closed state, the clamping-hammer contacts a respective force-measuring element and applies a pressure thereto.

In some embodiments, the apparatus can additionally comprise electronic circuitry for at least one of transmitting, displaying and recording, of a measurement of the respective force-measuring element.

In some embodiments, the pressure applied to the respective force-measuring element can be regulable by an adjustment of the clamping-hammer.

In some embodiments, every force-measuring element of the array of force-measuring elements can be aligned with a corresponding clamping-hammer. In some embodiments, every clamping-hammer of the array of clamping-hammers cam be aligned with a corresponding force-measuring element.

A method is disclosed, according to embodiments, for calibrating a surgical-arm motor-control unit that comprises an array of clamping-hammers aligned along an arm-receiving volume. The method comprises: (a) seating, in the arm-receiving volume, a calibration device comprising an elongate element and an array of force-measuring elements disposed thereupon; (b) closing a clamping-hammer to a respective closed state so as to contact a corresponding force-measuring element and to apply a pressure thereto; and (c) responsively to receiving information about a pressure from the corresponding force-measuring element, adjusting the clamping-hammer to modify the pressure.

In some embodiments, adjusting the clamping-hammer can include reorientating an exposed pressure-regulation portion of the clamping-hammer. In some embodiments, adjusting the clamping-hammer can include reorienting an exposed pressure-regulation portion of the clamping-hammer so as to adjust an orientation of an arm-contacting portion of the clamping-hammer. In some embodiments, adjusting the clamping-hammer can include reorienting an exposed pressure-regulation portion of the clamping-hammer so as to adjust a displacement of an arm-contacting portion of the clamping-hammer.

In some embodiments, modifying the pressure can includes at least one of: modifying the pressure to be within a predetermined range, and modifying the pressure to equal a predetermined value.

In some embodiments, modifying the pressure can includes modifying the pressure to be substantially equal to the pressure applied by a different clamping-hammer.

In some embodiments, the method can additionally comprise, subsequent to the adjusting of the clamping-hammer to modify the pressure: (i) seating a surgical arm in the arm-receiving volume, and (ii) closing the clamping-hammer to the respective closed state so as to contact the surgical arm and apply thereto a pressure modified by the adjusting of the clamping-hammer.

In some embodiments, it can be that, subsequent to the adjusting of the clamping-hammer and the seating of the surgical arm, a pressure applied to the surgical arm by the clamping-hammer is within ±20% of the modified pressure applied to the force-measuring element by the clamping-hammer.

In some embodiments, it can be that the adjusting of the clamping-hammer to modify the pressure is effective to regulate a force of engagement of a surgical-arm gear disposed on a surgical arm with a corresponding motor gear disposed within the arm-receiving volume.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIG. 1 is a schematic perspective view of a surgical device comprising two mechanical arms, according to embodiments of the present invention.

FIGS. 2A and 2B are, respectively, partial top and perspective schematic views of a motor-control unit of a surgical device, according to embodiments of the present invention.

FIG. 3 is a schematic illustration of a portion of a surgical arm, according to embodiments of the present invention.

FIG. 4 is a schematic illustration of a clamping-hammer applying a pressure to a contact area of a surgical arm, according to embodiments of the present invention.

FIGS. 5 and 6 are schematic perspective view of clamping-hammers in respective open and closed states, according to embodiments of the present invention.

FIGS. 7A, 7B and 7C are schematic side (elevation) views of a clamping-hammer adjustable by displacement of an arm-contacting portion of a clamping member, according to embodiments of the present invention.

FIG. 8 is a schematic side (elevation) views of a clamping-hammer adjustable by displacement of a clamping member, according to embodiments of the present invention.

FIG. 9 is a schematic side (elevation) views of a clamping-hammer adjustable by reorientation of a clamping member and/or of an arm-contacting portion, according to embodiments of the present invention.

FIGS. 10A, 10B and 10C are schematic and partial views of a motor-control unit comprising closure flaps, according to embodiments of the present invention.

FIG. 11 shows a schematic view of a portion of a surgical arm seated in an arm-receiving volume of a motor-control unit, according to embodiments of the present invention.

FIG. 12 is a schematic illustration of an apparatus for calibration clamping-hammers, according to embodiments of the present invention.

FIGS. 13 and 14 show flowcharts of method steps for calibrating a surgical-arm motor-control unit that comprises an array of clamping-hammers aligned along an arm-receiving volume, according to embodiments of the present invention.

FIG. 15 is a schematic side view of a clamping-hammer equipped with a respective force-measuring element, according to embodiments of the present invention.

FIG. 16 is a schematic perspective view of a surgical device comprising two mechanical arms and a calibration display, according to embodiments of the present invention.

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Note: Throughout this disclosure, subscripted reference numbers (e.g., 10₁ or 10_{A}) may be used to designate multiple separate appearances of elements of a single species, whether in a drawing or not; for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general. A double subscript may be used to indicate a member of an array, for example a clamping-hammer 135_{1A} is member A (e.g., of A, B, C) of a first array of clamping-hammers, the entire first array corresponding to a first arm-receiving volume 122₁ and a first surgical arm 102₁.

Embodiments disclosed herein relate to surgical devices using one or more surgical mechanical arms, i.e., articulated mechanical arms, using a plurality of different operating modes and/or a plurality of different input devices.

Whenever ‘arm’ is used herein or in the appended claims, it means an articulated, mechanical arm that is part of a surgical system or electrosurgical system and used for performing or helping to perform surgical (including, without limitation electrosurgical and imaging) actions inside a human subject's body. An articulated arm can also be considered flexible. An arm may include an end effector, which is used herein to mean a tool or device used in connection with surgery, electrosurgery, diagnosis or imaging when deployed within a human body. An end effector may be supplied as part of an arm, i.e., already mounted, mechanical attached and/or integrated with the power and communications conveyances of the arm; in some embodiments an arm an end effector may be provided separately for assembly and/or integration into a working unit before or even during a surgical operation, i.e., before insertion into a subject's body. A ‘surgical device’ as used herein means a device having one or more surgical mechanical arms and a motor unit or motor-control unit for housing and controlling the one or more arms.

In embodiments, it can be desirable for the motor-control unit to be arranged to engage surgical arms in such a way that (a) the arms are firmly seated and secured during operation, preferably with non-manipulable portions of the arm immobilized; (b) an appropriate force (or, equivalently pressure) is applied to the secured portion for transmittal, through the arm, to arm-control gears installed in the motor-control unit to ensure suitable contact, including meshing, between the control gears of the arm and the arm-control gears of the motor-control unit; and/or (c) the surgical arms can be efficiently and effectively replaced without disassembly of the motor-control unit, for example by a pre-surgical staff preparing the surgical device for use in an operation. Moreover, it can be desirable for a clamping arrangement deployed for meeting any or all of these goals to be readily adjustable and calibratable.

Clamping arrangements as disclosed herein include arrays of ‘clamping-hammers’ disposed to immobilize portions of surgical arms withing respective arm-receiving volumes of the motor-control unit. Clamping-hammers are mechanical assemblies affixed at their respective bases to the motor-control unit, preferably in proximity with, and aligned with, a respective arm-receiving volume designed to have secured therewithin a surgical arm. Each clamping-hammer includes a ‘clamping member’ which is biased, when in respective closed states, to have an arm-contacting portion of a clamping member contact, and apply a pressure to, a contact area of the arm.

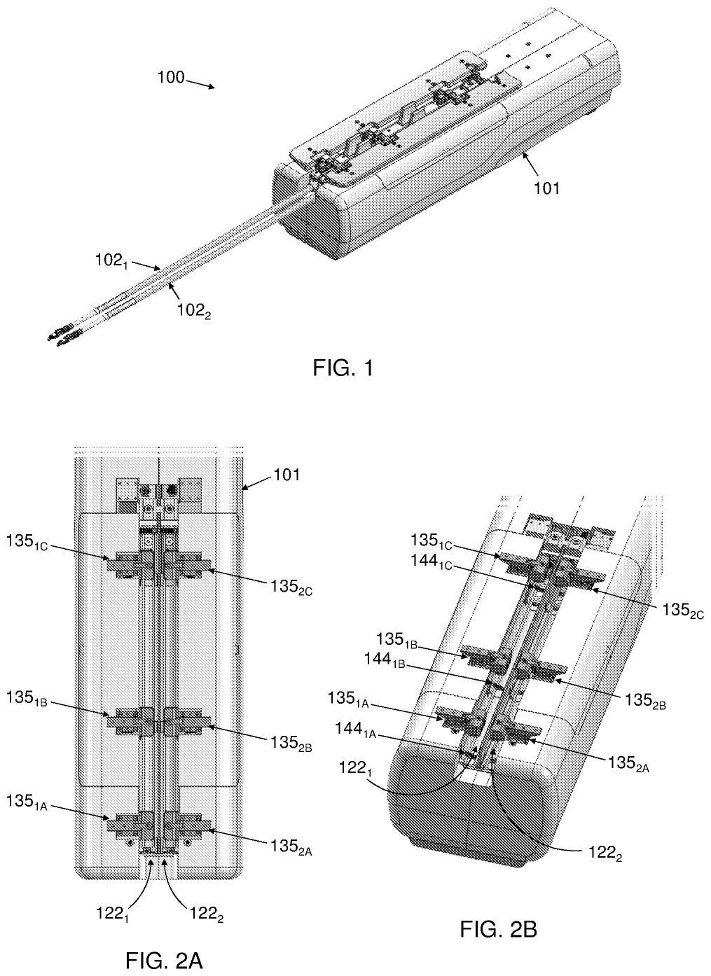

Referring now to the figures and in particular to FIG. 1, a surgical device 100 includes a motor-control unit 101 equipped with surgical arms 102. FIG. 1 illustrates a non-limiting example in which the surgical device includes two surgical arms 102₁, 102₂, but in other examples there can be a single surgical arm 102 or more than two.

FIGS. 2A and 2B show a motor-control unit 101 without surgical arms 102 installed in the motor-control unit 101. As seen in the schematic top view of FIG. 2A, the motor-control unit 101 includes two arm-receiving volumes 122₁, 122₂. Thus, the motor control unit 101 is configured to secure two surgical arms therewithin, as in the example of FIG. 1. Throughout this disclosure, two-arm motor-control units 101 are shown in various figures for illustration purposes and for improved clarity, but this is not necessarily indicative of any particular number of surgical arms 102 being preferred in implementation of the embodiments, and in fact the present embodiments are equally applicable to any suitable number of surgical arms 102 seated and secured in respective arm-receiving volumes 122. An array of clamping-hammers 135 according to embodiments is aligned longitudinally along and in proximity to each arm-receiving volume 122. In FIG. 2A, a first array of clamping-hammers [135_{1A}, 135_{1B}, 135_{1C}] is shown as being aligned with and in proximity to a first arm-receiving volume 122₁, and a second array of clamping-hammers [135_{2A}, 135_{2B}, 135_{2C}] is shown as being aligned with and in proximity to a second arm-receiving volume 122₂. This illustrates a particular example of use of the term ‘aligned’ in this disclosure in which the clamping-hammers 135 of a single array are equidistant from the arm-receiving volume 122; in other examples, aligned elements need not be strictly equidistant or equally spaced apart, etc., as long as they are substantially aligned, e.g., within 5% of being aligned, or within 10% of being aligned, or within 15% of being aligned or within 20% of being aligned or within 25% of being aligned or within 30% of being aligned.

The clamping-hammers 135 in FIGS. 2A and 2B are shown in respective closed states. As can be seen more clearly in FIG. 2B, being in the closed state means that a portion of the clamping-hammer 135 is in contact with the respective arm-receiving volume 122 such when a surgical arm 102 is at least partly seated in the arm-receiving volume 122, that part of the clamping-hammer 135 comes in contact with the surgical arm 102.

In embodiments, each arm-receiving volume 122 of a motor-control unit 101 includes an array of gears 144 which are typically spaced to meet corresponding gears (not shown) on a surgical arm 102. A gear on the surgical arm 102, for example, may be turned to actuate a movement, e.g., flexion or rotation, of a joint of the arm 102, and the gear on the surgical arm 102 is turned by the gear 144 in the motor-control unit 101. A proper contact and meshing of the gears 144 of the motor-control unit 101 with gears on the surgical arm 102 can therefore be beneficial to the precise operation of the surgical arm 102. The proper contact, in embodiments, is one that is accompanied by a predetermined force or, equivalently, pressure, being applied. The pressure applied by a closed, biased clamping-hammer 135 to the top, i.e., outward-facing-surface, of a surgical arm 102 can be regulated, as will be further discussed hereinbelow, and this pressure is most effectively transmitted through width of the arm 102 to a gear 144 of the motor-control unit 101 (and corresponding gear on the bottom, i.e., inward-facing surface, of the surgical arm 102) when the clamping-hammer 135 and motor-control-unit gear 144 are lined up vertically from top to bottom of the arm 102. In some embodiments, there can be more than one motor gear 144 in contact with the arm 102 at any one of the locations along the arm 102 contacted by a clamping-hammer 135. In some embodiments, a motor gear 144 can be disposed on an inner side wall of an arm-receiving portion 122 rather than on the bottom surface beneath the arm 102.

In the example of FIG. 2B, an array of clamping-hammers 135 is deployed at locations along the length of an arm-receiving portion 122 so as to align with, or correspond to, the array of gears 144. For example, each gear 144 of the first array of gears [144_{1A}, 144_{1B}, 144_{1C}] ‘lines up’ with a corresponding clamping-hammer 135 of the first array of clamping-hammers [135_{1A}, 135_{1B}, 135_{1C}]: gear 144_{1A} lines up with clamping-hammer 135_{1A}, and so on. Thus, when the arm first arm 102₁ is seated in the first arm-receiving volume 122 and the clamping-hammers 135 of the first array of clamping-hammers [135_{1A}, 135_{1B}, 135_{1C}] are all in respective closed states, the force/pressure applied to the arm 102 is transmitted to the gears 144 of the first array of gears [144_{1A}, 144_{1B}, 144_{1C}].

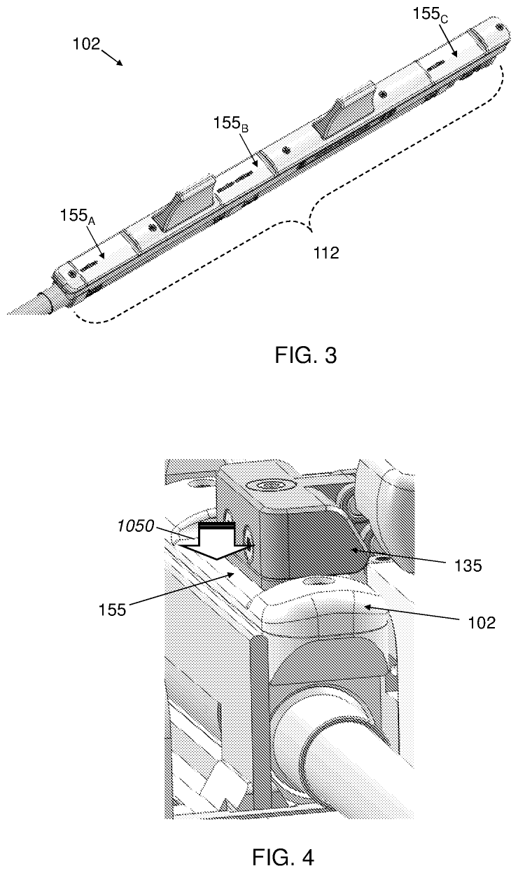

In some embodiments, a surgical arm 102 is adapted for receiving pressure from a closed clamping-hammer 135. Referring now to FIG. 3, a top surface of an exemplary surgical arm, i.e., the outward-facing surface when the arm 102 is seated in an arm-receiving volume 122 of a motor-control unit 101, comprises an array of contact areas [155_{A}, 155_{B}, 155_{C}] spaced and shaped, e.g., having a flat surface, to receive the pressure from a clamping-hammer 135 biased to apply a pressure when in a closed state. The applied pressure is due to a force, indicated by arrow 1050, in FIG. 4, exerted by the clamping-hammer 135 on the contact area 155. In embodiments, the clamping-hammer is biased to apply a force (1050) when in the closed state shown in FIG. 4.

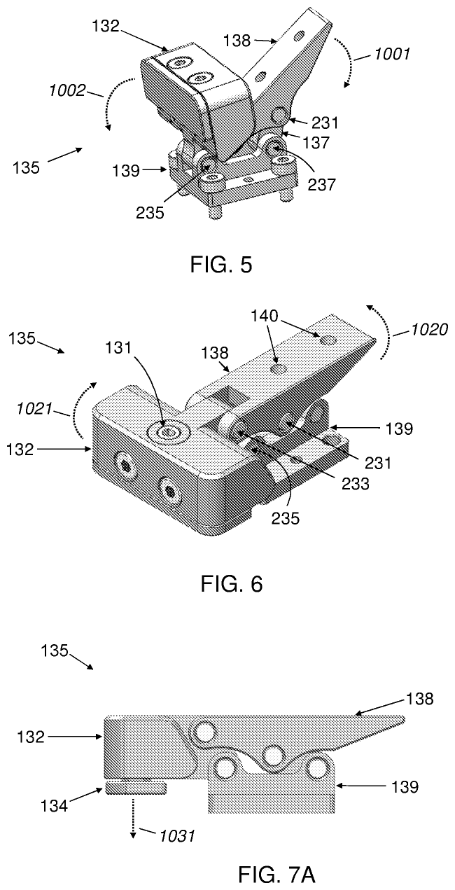

Reference is made to FIGS. 5, 6, 7A, 7B, 8 and 9, all of which are schematic illustrations of clamping-hammers 135 according to embodiments.

In embodiments, a clamping-hammer includes a base member 139, a lever member 138, and a clamping member 132. A base member 139 is configured for installation on the motor-control unit 101, and as such affixes the clamping-hammer 135 in place. A clamping member 132 is that portion of the clamping-hammer 135 which contacts a surgical arm 102 so as to apply a pressure thereto, and keeps the arm 102 in place within an arm-receiving portion 122 of the motor-control unit 101 such that, for example, gears of the surgical arm 102 are in proper geared contact with gears 144 of the motor-control unit 101. A clamping member 132 includes an arm-contacting portion 134 on the bottom of the clamping member, where ‘bottom’ means towards the arm-receiving portion 122 or towards a surgical arm 102 seated in the arm-receiving portion 122. In some embodiments, the arm-contacting portion 134 is an integral portion of the clamping member 132. In some embodiments, the arm-contacting portion 134 is a separable, e.g., attachable/detachable, member coupled to the bottom of the clamping member 132. In some of those embodiments in which the arm-contacting portion 134 is a distinct member coupled to the bottom of the clamping member 132, the arm-contacting portion 134 is displaceable and/or re-orientable separately from and/or relative to, the clamping member 132, i.e., relative to the portion of the clamping member 132 that does not include the separable arm-contacting portion 134.

A lever member 138 operates as a lever to move the clamping member 132 from an open position to a closed position and vice versa. Note: the terms ‘state’ and ‘position’ are used interchangeably herein to describe orientations of the clamping-hammers 135 and/or of components thereof. The closed position or state is that orientation in which the clamping member is in a ‘proximal’ position with respect to, i.e., closer to, the arm-receiving portion 122 or the arm 102 itself if present. The open position or state is that orientation in which clamping member is in a ‘distal’ position with respect to, i.e., further from, the arm-receiving portion 122 or the arm 102 itself if present.

The closing movement is illustrated schematically in FIG. 5, wherein arrow 1001 indicates a downward pivoting movement of the lever member 137 and arrow 1002 indicates the downward pivoting movement of the clamping member 132, the latter movement being leveraged by the downward pivoting movement of the lever member 138. The closing movement is effective to move the clamping-hammer 135 from the open state illustrated in FIG. 5 to the closed state illustrated in FIG. 6. The corresponding opening movement is illustrated in FIG. 6, wherein arrow 1020 indicates an upward pivoting movement of the lever member 138 and arrow 1021 indicates the upward pivoting movement of the clamping member 132, the latter movement being leveraged by the upward pivoting movement of the lever member 138. The opening movement is effective to move the clamping-hammer 135 from the closed state illustrated in FIG. 6 to the open state illustrated in FIG. 5.

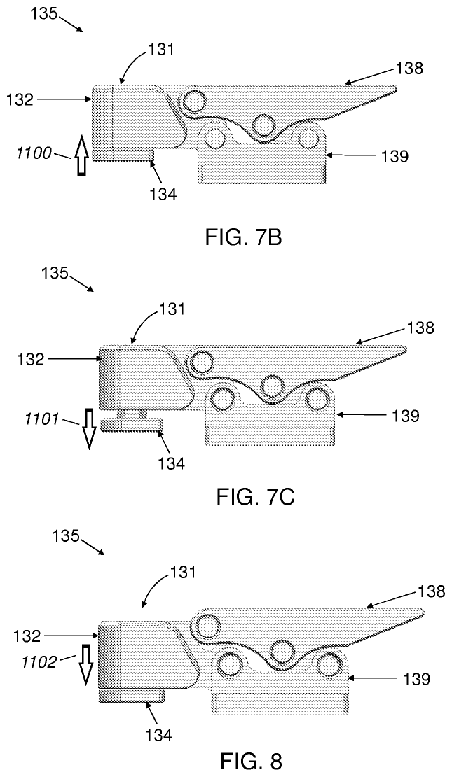

In embodiments, the lever member 138 is pivotably attached to both the base member 139 and to the clamping member 132. In the non-limiting example shown in FIG. 5, the lever member 138 is indirectly pivotably attached to the base member 139 and directly pivotably attached to a biasing member 137 by pivot member 231; the biasing member 137 is pivotably attached to the base member 139 by pivot member 237. The biasing member 137 is configured to bias the clamping member 132 into the closed position such that the clamping member 132 applies a downward force—where ‘downward’ is the direction indicated in FIG. 7A by arrow 1031. Thus, the clamping member 132, when in the closed position, is biased to apply a force to a contact area 155 of a surgical arm 102. The skilled artisan will understand that using a biasing member 137 is not the only way to bias the clamping-hammer 135, and particularly the clamping member 132, to apply such force; in other configurations other mechanical arrangements can be used, and in still other configurations, a spring mechanism (not shown) can be used to provide the biasing force. In the exemplary configuration illustrated schematically in FIG. 6, the clamping member is pivotably attached to both the base member 139 (by pivot member 235) and the lever member 138 (by pivot member 233). The scope of the embodiments is in no way limited by the specific design of the illustrated configuration. In another non-limiting example (not shown), the clamping member 132 is pivotably attached to a biasing element (not shown) and only indirectly to either the lever member 138 or the base member 139.

According to embodiments, it can be desirable to provide a user with the ability to regulate the amount of force or pressure (indicated by arrow 1031) applied to the surgical arm 102 by each of the clamping-hammers 135. As shown in FIG. 6, a regulating member 131 is provided for that purpose. As configured in the example of FIG. 6, the regulating member 131 includes a threaded element, e.g., a threaded bolt or screw, where regulating the pressure of the clamping member 132 is accomplished by rotating the threaded element of the regulating member 131. Only the exposed top of the regulating member 131 is shown in the figures. In other examples, the regulating member 131 can include any suitable combination of mechanical elements such as buttons and levers for regulating the pressure. In the example of FIG. 7A, the arm-contacting portion 134 includes a separate pressure member of adjustable height or orientation. As indicated schematically by the arrow 1100 of FIG. 7B, the arm-contacting portion 134 is lowerable by rotation of the regulating-member threaded element 131. Based on the biasing design of the clamping-hammers 135 as discussed hereinabove, lowering the arm-contacting portion 134 relative to the remainder of the clamping member 132 increases the pressure applied to the surgical arm 102. As indicated schematically by the arrow 1101 of FIG. 7C, the arm-contacting portion 134 is raisable by rotation of the regulating-member threaded element 131. Based on the biasing design of the clamping-hammers 135 as discussed hereinabove, raising the arm-contacting portion 134 relative to the remainder of the clamping member 132 decreases the pressure applied to the surgical arm 102.

FIG. 8 illustrates a configuration in which the clamping member 132 itself and not just an arm-contacting portion 134, can be raised and lowered by a regulating member 131 in order to decrease and increase, respectively, the pressure applied by the clamping-hammer 135 to the surgical arm 135 seated in the arm-receiving portion 122 of the motor-control unit 101. This approach is suitable, for example, in designs in which the arm-contacting portion 134 is an integral part of the clamping member 132 and not a separable part. Arrow 1102 indicates a lowering (downward) movement of the clamping member 132 responsive to an adjustment made by regulating member 131, e.g., a rotation of a threaded element.

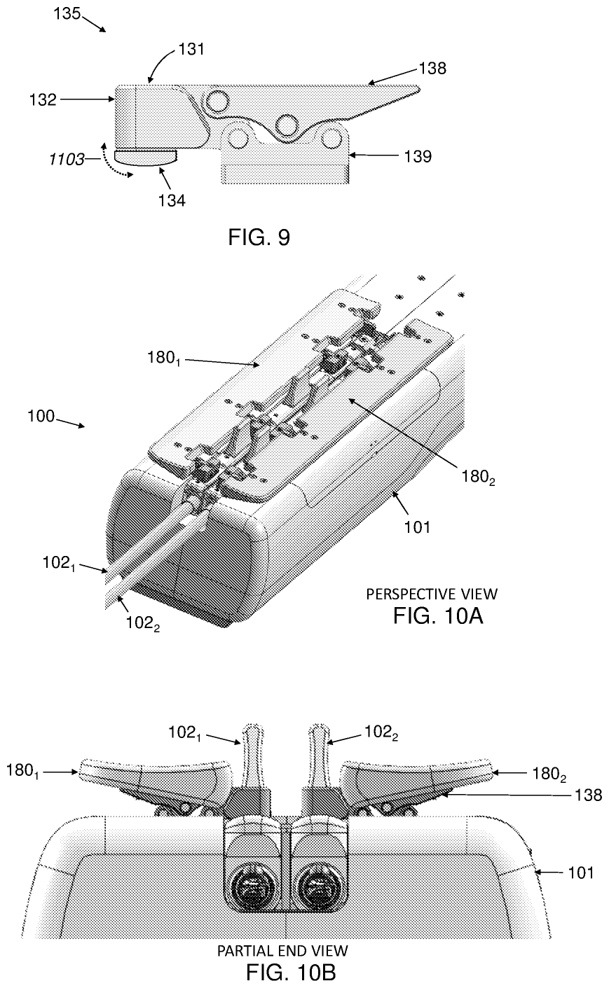

FIG. 9 illustrates a configuration in which an arm-contacting portion 134, e.g., embodied as a separate pressure member, has a curved face for contacting the surgical arm 102. The clamping member 132 is arranged to pivot when regulated by reorienting the regulating member 131, as indicated by arrow 1103, so as to increase or decrease pressure on the arm 102 in accordance with the specific design and direction of regulation.

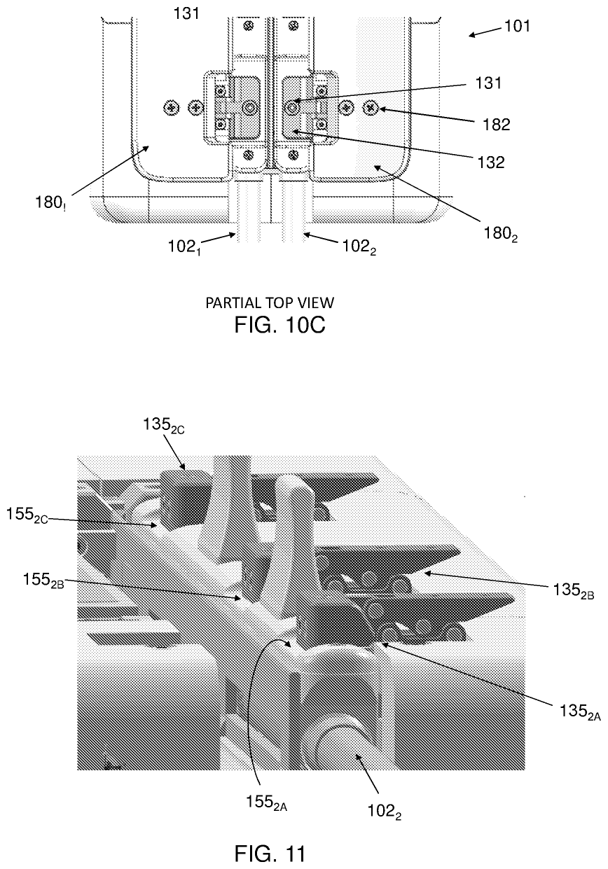

Reference is now made to FIGS. 10A, 10B, 10C and 11, all of which disclose features related to employing a closing flap in conjunction with clamping-hammers.

According to embodiments, it can be advantageous to provide a closure flap 180 to a motor-control unit 101 for simultaneous opening and closing of multiple clamping-hammers 135. In the example of FIG. 10A, a first closure flap 180₁ is secured to all three clamping-hammers 135 of the first array of clamping-hammers [135_{1A}, 135_{1B}, 135_{1C}], and a first closure flap 180₂ is secured to all three clamping-hammers 135 of the second array of clamping-hammers [135_{2A}, 135_{2B}, 135_{2C}]. FIG. 10B shows a partial end view of the motor-control unit 101 with the closure flaps 180₁, 180₂ coupled to lever members 138 of the clamping-hammers 135. FIG. 10C is a partial top view of the motor-control unit 101. Referring again to FIG. 6, a lever member 138 of a clamping-hammer 135 can include one or more flap-attachment points 140 for coupling to a closure flap 180. The flap-attachment points 140 of the non-limiting example of FIG. 6 are embodied by simple holes through which attachment hardware, e.g., screws, bolts or rivets can be inserted for firm attachment with corresponding features on the closing flap 180. As shown in FIG. 10C, a closing flap 180 can include attachment elements 182 that correspond to the flap-attachment points 140 on the lever members 138. The coupling of closing flaps 180 to clamping-hammers 135 affords a simplified procedure for opening and closing clamping-hammers: pivoting a closure flap 180 to a closed position (e.g., as shown in FIGS. 10A, 10B and 10C) is effective to place all of the clamping-hammers 135 (e.g., an entire array of clamping-hammers 135) coupled to the closure flap 180 in respective closed positions, i.e., positions proximal to the surgical arm 102. Similarly, pivoting a closure flap 180 to an open position (not shown) is effective to place all of the coupled clamping-hammers 135 (e.g., an entire array of clamping-hammers 135) coupled to the closure flap 180 in respective open positions, i.e., positions distally displaced from the surgical arm 102.

FIG. 10C illustrates a preferred design in which regulating members 131 disposed on clamping members 132 are ‘exposed’ when the clamping members 135 and closure flaps 180 are all in respective closed positions. The term ‘exposed’ as used herein means both ‘not covered’, e.g., by closure flaps, and disposed on an exposed surface for access by a user to the extent necessary to regulate pressure applied by a clamping-hammer 135 to a surgical arm 101.

According to some embodiments, an array of clamping-hammers [135_{A}. . . 135ₙ] includes at least three clamping-hammers, as illustrated in FIG. 11. As is known in the art, when applying a force at three or more points, e.g., by three or more clamping-hammers, care must be taken to ensure to equalize or balance the forces. In the instant example, if the forces are not regulated, there can be a situation where there are two (or) more ‘dominant’ clamping-hammers 135 that could render one or more other clamping-hammers 135 ineffective in transmitting a pressure through the surgical arm to a corresponding motor-control-unit gear 144. Therefore, the three (or more) clamping-hammers 135 are jointly regulable to apply substantially equal pressures to corresponding contact areas 155 of the surgical arm 102. “Substantially” in this context means within 5% of each other, or within 10% of each other, or within 15% of each other, or within 20% of each other.

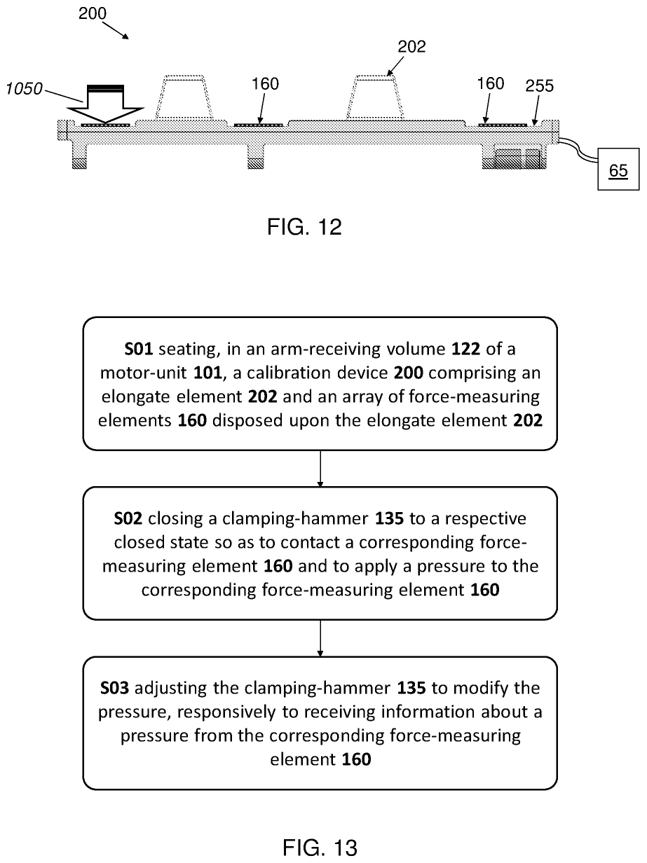

In embodiments, pressure applied to surgical arm 102, e.g., at a contact area 155, can be regulated, e.g., by a reorientation of the regulating member 131, with an instrumented device seated in the arm-receiving volume 122. An example of a suitable instrumented device is the exemplary calibration apparatus 200 shown in FIG. 12. The calibration apparatus 200 comprises an elongate element 202 equipped with force-measuring elements 160. Force-measurement elements 160 can comprise load cells, strain gages, and/or any other instruments for measuring forces and/or pressures as are known in the art. As can be seen in FIG. 12, the calibration apparatus 200 emulates a surgical arm 102 in whatever aspects of form, shape, weight, density and hardness are deemed beneficial by the designer of the calibration apparatus 200. The force-measuring elements 160 are suitably located at portions of the calibration apparatus 200 where clamping-hammers 135 will apply respective pressures (indicated, as an example, by the arrow 1050 in FIG. 12) when the calibration apparatus 200 is seated in the arm-receiving volume 122. Preferably, a force-measuring element 160 is provided to correspond to each one of the clamping-hammers 135 in an array of clamping-hammers [135_{A}. . . 135ₙ] such that there is a force-measurement element 160 for each clamping-hammer, and, similarly a clamping-hammer 135 for each force-measuring element 160. In some embodiments, the force-measuring elements 160 are located in contact areas 255 that emulate contact areas 155 of surgical arms 102. A calibration apparatus 200 can be used in conjunction with the regulating members 131 of clamping-hammers 135 in setting applied pressures—for example, to match a predetermined value or to fall within a predetermined range of values. In some embodiments, the pressure at one clamping-hammer 135 is regulated to be substantially equal to the pressure applied by one or more different clamping-hammer 135, as discussed hereinabove with respect to clamping-hammer arrays [135_{A}. . . 135ₙ] comprising three or more clamping-hammers 135. The calibration apparatus 200, in some embodiments, comprises electronic circuitry 65 for at least one of transmitting, displaying and recording, of a measurement of the respective force-measuring elements 160. For the present disclosure, “electronic circuitry” is intended broadly to describe any combination of hardware, software and/or firmware. Electronic circuitry may include any executable code module (i.e. stored on a computer-readable medium) and/or firmware and/or hardware element(s) including but not limited to field programmable logic array (FPLA) element(s), hard-wired logic element(s), field programmable gate array (FPGA) element(s), and application-specific integrated circuit (ASIC) element(s). Any instruction set architecture may be used including but not limited to reduced instruction set computer (RISC) architecture and/or complex instruction set computer (CISC) architecture. Electronic circuitry may be located in a single location or distributed among a plurality of locations where various circuitry elements may be in wired or wireless electronic communication with each other.

A method is disclosed, according to embodiments, for calibrating a surgical-arm motor-control unit 101 that comprises an array of clamping-hammers [135_{A}. . . 135ₙ] aligned along an arm-receiving volume 122. Calibrating the motor-control unit 101 in this context is equivalent to calibrating the clamping-hammers 135. As shown in the flowchart of FIG. 13, the method comprises any or all of the following steps:
Step S01: seating, in an arm-receiving volume 122 of a motor-unit 101, a calibration device 200 comprising an elongate element 202 and an array of force-measuring elements 160 disposed upon the elongate element 202;
Step S02: closing a clamping-hammer 135 to a respective closed state so as to contact a corresponding force-measuring element 160 and to apply a pressure to the corresponding force-measuring element 160; and
Step S03: adjusting the clamping-hammer 135 to modify the pressure, responsively to receiving information about a pressure from the corresponding force-measuring element 160.

Adjusting the clamping-hammer 135 to modify the pressure can be effective to regulate a force of engagement of a surgical-arm gear disposed on a surgical arm 102 with a corresponding motor gear 144 disposed within the arm-receiving volume 122 of a motor-control unit 101. In embodiments, adjusting the clamping-hammer 135 includes reorientating an exposed pressure-regulation portion 131 of the clamping-hammer 135. In embodiments, adjusting the clamping-hammer 135 includes reorienting an exposed pressure-regulation portion 131 of the clamping-hammer 135 so as to adjust an orientation and/or displacement of the arm-contacting portion 134 of the clamping-hammer 135. In some embodiments, modifying the pressure can be to match a predetermined value or to fall within a predetermined range of values, or to substantially equal a pressure applied by one or more different clamping-hammers 135.

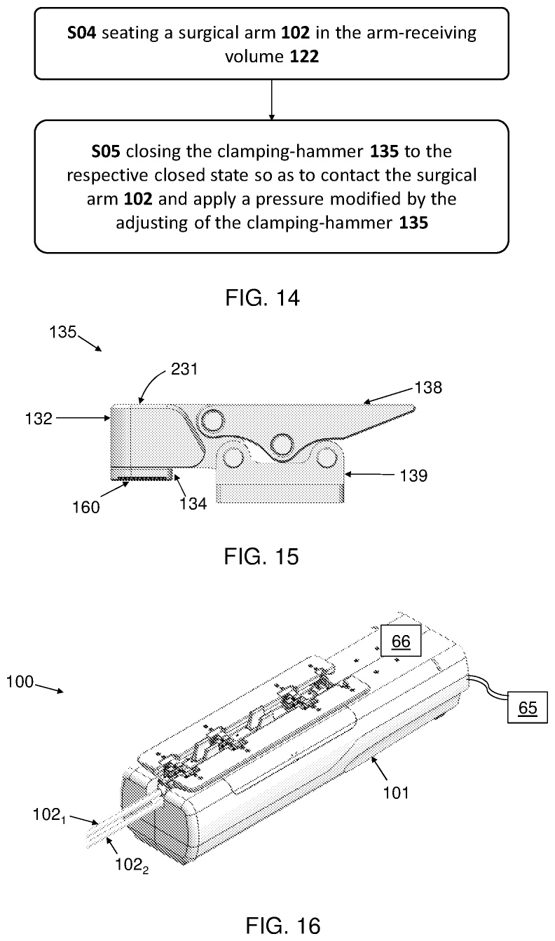

In some embodiments, the method additionally comprises one or more of the method steps shown in the flowchart of FIG. 14:
Step S04: seating a surgical arm 102 in the arm-receiving volume 122; and
Step S05: closing the clamping-hammer 135 to the respective closed state so as to contact the surgical arm 102 and apply a pressure modified by the adjusting of the clamping-hammer 135.

If the calibration apparatus effectively emulates a surgical arm 102 for the purpose of calibrating the clamping-hammers 135, then subsequent to the adjusting of the clamping-hammers 135 (Step S03) and the seating of the surgical arm 102 (Step S05), a pressure applied to the surgical arm 102 by any calibrated clamping-hammer 135 is within ±20% of the modified pressure that was applied by the clamping-hammer 135 to the force-measuring element 160 of the calibration apparatus 200 at the end of Step S03.

The scope of the above methods extends to carrying them out using an instrumented surgical arm 102 as the calibration apparatus 200, i.e., by equipping a functional surgical arm 102 with force-measuring elements 160 and suitable electronic circuitry 65.

FIG. 15 illustrates a clamping-hammer 135 equipped with a force-measuring element 160. This clamping-hammer design can be effective in performing calibrations without the use of a special-purpose calibration apparatus 200 and can be used with surgical arms 102 that are not specially instrumented for force measurements. The clamping-hammer 135 of FIG. 15 can be used effectively in conjunction with the motor-control unit 101 of FIG. 16, which includes electronic circuitry 65 for processing force measurements from the clamping-hammers. In an example, the force-measuring elements 160 measure forces applied by the clamping-hammers 135 to a surgical arm 102, and perform one or more of the following steps, e.g., in conjunction with the electronic circuitry 65: (i) display information about the force measurements using display 66 on the motor-control unit 101, where the information can include, for example, measurement data and/or a go/no go calibration indication such as a red or green light indication; (ii) disable or enable the operation of the motor-control unit 101 and/or of the surgical arms 102 until a calibration is completed; and (iii) automatically calibrate the pressures applied by the clamping-hammers 135 using automated pressure-regulating members 231, installed in the clamping-hammers and configured to receive information about calibration from the electronic circuitry 65.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1. A surgical apparatus comprising a motor-control unit for controlling one or more surgical arms, the motor-control unit comprising, for each one of the one or more surgical arms:
i. an arm-receiving volume shaped to receive a portion of a respective one of the one or more surgical arms, and
ii. an array of clamping-hammers aligned with the arm-receiving volume and configured for applying pressure to said portion of the surgical arm to thereby secure said portion in said volume, each clamping-hammer comprising:
a. a clamping member configured for displacement with respect to said volume at least between a distal position spaced from said arm-receiving volume and a proximal position adjacent to said arm-receiving volume; and
b. a pressure member fitted to the clamping member and configured for coming in contact with a respective contact area of said portion of the surgical arm, wherein said pressure member is configured to displace with respect to the clamping member, thereby allowing regulation of the pressure applied to said respective contact area.

2. The surgical apparatus of claim 1, additionally comprising a closure flap in communication with one or more clamping members, wherein pivoting the closure flap to a closed position is effective to place the one or more clamping members in respective proximal positions.

3. The surgical apparatus of claim 2, wherein (i) each clamping hammer further comprises a regulating member fitted to the clamping member and configured to regulate the displacing of a respective pressure member, and (ii) when the one or more clamping members are in respective proximal positions and the closure flap is in the closed position, regulating members respective of the one or more clamping members are exposed.

4. The surgical apparatus of claim 3, wherein the displacing of a pressure member is regulable by reorientation of the respective regulating member

5. (canceled)

6. (canceled)

7. The surgical apparatus of claim 3, wherein the pressure applied to said portion of the surgical arm is set by a reorientation of the regulating member performed with an instrumented calibration apparatus seated in the arm-receiving volume.

8. The surgical apparatus of claim 1, wherein the array of clamping-hammers includes at least three clamping-hammers, and the at least three clamping-hammers are jointly regulable to apply substantially equal pressures to said portion of the surgical arm at respective contact areas.

9. (canceled)

10. The surgical apparatus of claim 1, wherein the regulation of the pressure applied to a respective contact area is effective to regulate a force of engagement of a surgical-arm gear of the respective surgical arm with a corresponding motor gear disposed within the arm-receiving volume.

11. A motor-control unit for controlling one or more surgical arms, the motor unit comprising, for each one of the one or more surgical arms:
a. an arm-receiving volume shaped to receive a portion of the respective surgical arm, and
b. an array of clamping-hammers aligned along the arm-receiving volume, each clamping-hammer being orientable to a respective closed state and biased, when in said respective closed state, to contact a surgical arm seated in the arm-receiving volume and to apply a pressure thereto, wherein the pressure is regulable by reorienting a pressure-regulating portion of the clamping-hammer

12. The motor-control unit of claim 11, wherein each clamping-hammer of the array of clamping-hammers comprises:
i. a base member anchored to the motor-control unit,
ii. a clamping member pivotably attached to the base member, and
iii. a lever member pivotably attached to each of the base member and the clamping member, and biased, when in a closed state, to transfer a clamping force to the clamping member, the clamping force being effective, when the respective surgical arm is seated in the arm-receiving volume, to cause an arm-contacting portion of the clamping member to contact the surgical arm and to apply a pressure thereto.

13. (canceled)

14. (canceled)

15. (canceled)

16. (canceled)

17. (canceled)

18. (canceled)

19. The motor-control unit of claim 11, wherein the pressure applied to the surgical arm is set by a reorientation of the pressure-regulating portion performed with an instrumented calibration apparatus seated in the arm-receiving volume.

20. The motor-control unit of claim 11, wherein the array of clamping-hammers includes at least three clamping-hammers, and the at least three clamping-hammers are jointly regulable to apply substantially equal pressures to the surgical arm.

21. The motor-control unit of claim 11, further comprising, for each one of the one or more surgical arms, an array of gears arranged in the arm-receiving volume to be in geared contact with corresponding surgical-arm gears of the surgical arm, wherein each clamping-hammer of at least a plurality of clamping-hammers is aligned with a corresponding motor-unit gear in the arm-receiving volume.

22. The motor-control unit of claim 11, wherein the regulation of the pressure applied to the surgical arm by each clamping-hammer is effective to regulate a force of engagement of a surgical-arm gear of the surgical arm with a corresponding motor gear disposed within the arm-receiving volume.

23. An apparatus for use with a surgical-arm motor-control unit that comprises an array of clamping-hammers aligned along an arm-receiving volume, each clamping-hammer being closable to a respective closed state so as to contact a surgical arm seated in the arm-receiving volume and to apply a pressure thereto, the apparatus comprising:
a. an elongate element shaped to be seated in the arm-receiving volume; and
b. an array of force-measuring elements, wherein for at least one of the clamping-hammers, when the apparatus is seated in the arm-receiving volume and the clamping-hammer is in its respective closed state, the clamping-hammer contacts a respective force-measuring element and applies a pressure thereto.

24. (canceled)

25. (canceled)

26. (canceled)

27. (canceled)

28. A method of calibrating a surgical-arm motor-control unit that comprises an array of clamping-hammers aligned along an arm-receiving volume, the method comprising:
a. seating, in the arm-receiving volume, a calibration device comprising an elongate element and an array of force-measuring elements disposed thereupon;
b. closing a clamping-hammer to a respective closed state so as to contact a corresponding force-measuring element and to apply a pressure thereto; and
c. responsively to receiving information about a pressure from the corresponding force-measuring element, adjusting the clamping-hammer to modify the pressure.

29. The method of claim 28, wherein adjusting the clamping-hammer includes reorientating an exposed pressure-regulation portion of the clamping-hammer.

30. (canceled)

31. (canceled)

32. The method of claim 28, wherein modifying the pressure includes at least one of: modifying the pressure to be within a predetermined range, and modifying the pressure to equal a predetermined value.

33. (canceled)

34. The method of claim 28, additionally comprising, subsequent to the adjusting of the clamping-hammer to modify the pressure: (i) seating a surgical arm in the arm-receiving volume, and (ii) closing the clamping-hammer to the respective closed state so as to contact the surgical arm and apply thereto a pressure modified by the adjusting of the clamping-hammer.

35. The method of claim 28, wherein, subsequent to the adjusting of the clamping-hammer and the seating of the surgical arm, a pressure applied to the surgical arm by the clamping-hammer is within ±20% of the modified pressure applied to the force-measuring element by the clamping-hammer.

28. The method of claim 28, wherein the adjusting of the clamping hammer to modify the pressure is effective to regulate a force of engagement of a surgical-arm gear disposed on a surgical arm with a corresponding motor gear disposed within the arm-receiving volume.
